(19) United States
(12) Patent Application Publication
(10) Pub. No.: US 2018/0303415 A1
(43) Pub. Date: Oct. 25, 2018

(54) WEARABLE APPARATUS AND AUDIO-VIDEO RECORDING METHOD THEREOF

(71) Applicant: Singular Wings Medical Co., Ltd., Hsinchu County (TW)

(72) Inventor: Wei-Chung Lee, Hsinchu County (TW)

(73) Assignee: Singular Wings Medical Co., Ltd., Hsinchu County (TW)

(21) Appl. No.: 15/629,745

(22) Filed: Jun. 22, 2017

(30) Foreign Application Priority Data
Apr. 24, 2017 (TW) 106113660

(51) Int. Cl. A61B 5/00; G06F 19/00

(52) U.S. Cl. CPC A61B 5/6801; G06F 13/00; A61B 5/00; G06F 19/3418

(57) ABSTRACT

The invention provides a wearable apparatus and an audio-video recording method thereof. The wearable apparatus includes a sensing module, an audio-video (AV) module and a control module. The sensing module senses a physiological information of a user and an environment information of an environment where the user is located. The AV module captures a video and an audio around the user as an AV signal. The control module compares the physiological information and the environment information with at least one reference information. When at least one of the physiological information and the environment information matches one of the at least one reference information, the control module determines that an event occurs, and preserves and stores the AV signal within a period of time before and after a time point at which the event occurs.

### CROSS REFERENCE TO RELATED APPLICATION

This application claims the priority benefit of Taiwan application serial no. 106113660, filed on Apr. 24, 2017. The entirety of the above-mentioned patent application is hereby incorporated by reference herein and made a part of specification.

### BACKGROUND OF THE INVENTION

### 1. Field of the Invention

The invention relates to a wearable apparatus, and particularly relates to a wearable apparatus capable of automatically recording video and audio and an audio-video recording method thereof.

### 2. Description of Related Art

With advancements in technologies, various wearable apparatuses have been developed. A part of the wearable apparatuses can execute certain basic functions, such as timing, calculating, and game-playing functions. Another part of the wearable apparatuses can execute applications (APP), common programs or even operating systems (OS), and these wearable apparatuses are applied to the biomedical fields for health care or physical fitness tracking. In addition to abovementioned functions, how to further realize enhancement and diversification for the functions of the wearable apparatus as well as allowing the wearable apparatus to provide information that helps medical staffs conduct appropriate treatments in response to a specific condition occurred on a user of the wearable apparatus or helps in determining and clarifying attribution of responsibility for said specific condition are targets for people skilled in the art to devote to.

### SUMMARY OF THE INVENTION

The invention provides a wearable apparatus and an audio-video recording method thereof, which can preserve and store an information within a period of time before and after a time point at which a specific event occurs, so as to facilitate appropriate treatments or determining and clarifying attribution of responsibility in subsequent processes. In particular, the stored information can include the audio-video signal around the user, the physiological information of the user and the environment information of the environment where the user is located.

The wearable apparatus of the invention includes a sensing module, an audio-video module and a control module. The sensing module is configured to sense a physiological information of a user and an environment information of an environment where the user is located. The audio-video module captures a video and an audio around the user as an audio-video signal. The control module is coupled to the sensing module for receiving the physiological information and the environment information, and is coupled to the audio-video module for receiving the audio-video signal. The control module compares the physiological information and the environment information with at least one reference information. When at least one of the physiological information and the environment information matches one of the at least one reference information, the control module determines that an event occurs, and preserves and stores the audio-video signal within a period of time before and after a time point at which the event occurs.

In an embodiment of the invention, when aforesaid event occurs, the control module further preserves and stores the physiological information and the environment information within the time period before and after the time point at which the event occurs. The environment information includes at least one of a value of gravity acceleration taken by the user, a temperature value, a humidity value, an air pressure value, an ultraviolet value and a particulate matter (PM2.5) value in the environment where the user is located. The physiological information includes at least one of a body temperature value, a blood pressure value, a blood glucose value, a blood oxygen value, a brain wave value, an electrocardiogram value, a heart rate value, an electromyography value, a respiratory rate value, a breath chemical composition value and a sweat secretion value of the user.

In an embodiment of the invention, the control module determines that the event does not occur when neither of the physiological information and the environment information matches the at least one reference information, and the control module controls the audio-video module to perform a cyclic audio-video recording so as to perform a cyclic storage for the received audio-video signal.

In an embodiment of the invention, the wearable apparatus further includes a storage module. The storage module is coupled to the control module and the audio-video module. When the event occurs, the control module controls the audio-video module to store the audio-video signal within the period of time before and after the time point at which the event occurs into the storage module, and the control module stores the physiological information and the environment information received within the period of time before and after the time point at which the event occurs into the storage module.

In an embodiment of the invention, the wearable apparatus further includes a communication module. The communication module is coupled to the storage module, and configured to transmit the audio-video signal, the physiological information and the environment information stored in the storage module to an external electronic apparatus.

In an embodiment of the invention, the wearable apparatus further includes a warning module. The warning module is coupled to the control module. When the event occurs, the control module controls the warning module to send out a warning signal.

In an embodiment of the invention, the control module further includes a data storage module. The data storage module is configured to store the at least one reference information. The control module updates the at least one reference information based on a requirement of the user.

The invention provides an audio-video recording method of a wearable apparatus, which includes the following steps. A video and an audio around a user are captured as an audio-video signal by an audio-video module of the wearable apparatus. A physiological information of the user and an environment information of an environment where the user is located are sensed by a sensing module of the wearable apparatus. The physiological information and the environment information are compared with at least one reference information by a control module of the wearable apparatus, and whether at least one of the physiological information and the environment information matches one of the at least one reference information is determined and a comparison result is obtained. It is determined that an event occurs if the comparison result is yes, and the audio-video signal within a period of time before and after a time point at which the event occurs is preserved and stored by the control module.

In an embodiment of the invention, the audio-video recording method of the wearable apparatus further includes: preserving and storing the physiological information and the environment information within the period of time before and after the time point at which the event occurs by the control module when the event occurs.

In an embodiment of the invention, the audio-video recording method of the wearable apparatus further includes: determining that the event does not occur when neither of the physiological information and the environment information matches the at least one reference information, and controlling the audio-video module by the control module to perform a cyclic audio-video recording so as to cyclically store the received audio-video signal.

Based on the above, once a specific event occurs on the user of the wearable apparatus, the wearable apparatus and the audio-video recording method thereof of the invention are capable of preserving and storing the related information within the period of time before and after the time point at which the specific event occurs, so as to facilitate appropriate treatments or determining and clarifying attribution of responsibility in subsequent processes.

To make the above features and advantages of the disclosure more comprehensible, several embodiments accompanied with drawings are described in detail as follows.

### BRIEF DESCRIPTION OF THE DRAWINGS

The accompanying drawings are included to provide a further understanding of the invention, and are incorporated in and constitute a part of this specification. The drawings illustrate embodiments of the invention and, together with the description, serve to explain the principles of the invention.

FIG. 1 illustrates a circuit block diagram of a wearable apparatus according to an embodiment of the invention.

FIG. 2 illustrates a circuit block diagram of a wearable apparatus according to another embodiment of the invention.

FIG. 3 is a flowchart illustrating steps in an audio-video recording method according to an embodiment of the invention.

FIG. 4 is a flowchart illustrating steps in an audio-video recording method according to another embodiment of the invention.

### DESCRIPTION OF THE EMBODIMENTS

Reference will now be made in detail to the present preferred embodiments of the invention, examples of which are illustrated in the accompanying drawings. Wherever possible, the same reference numbers are used in the drawings and the description to refer to the same or like parts.

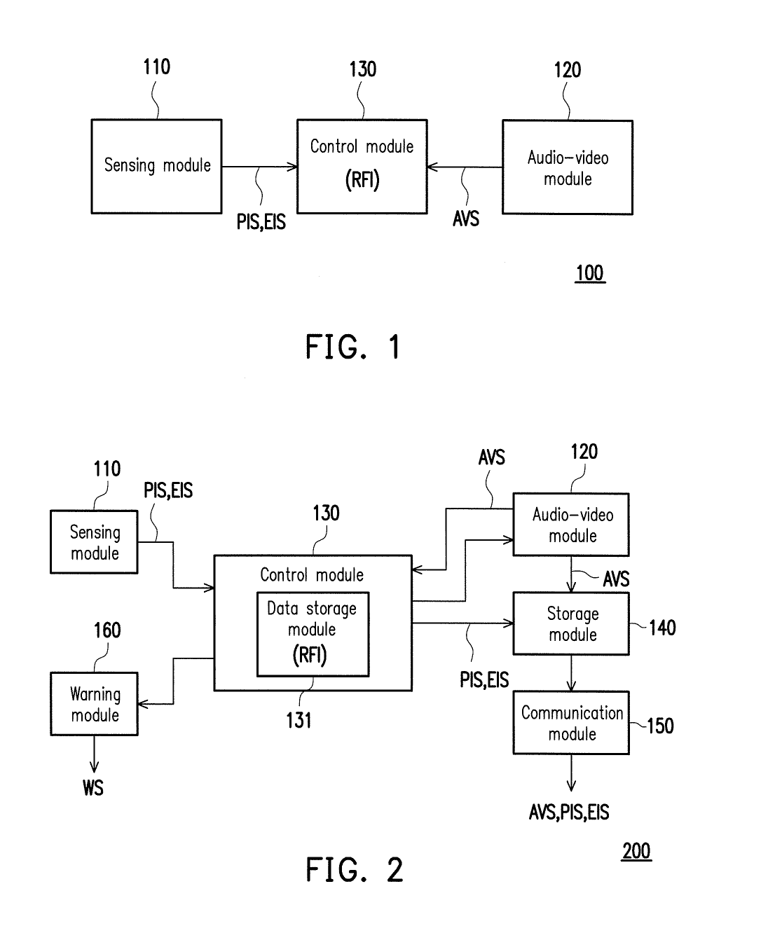

FIG. 1 illustrates a circuit block diagram of a wearable apparatus 100 according to an embodiment of the invention. With reference to FIG. 1, in the present embodiment, the wearable apparatus 100 may be a wearable electronic apparatus realized in a user-wearable manner, including a tag, a wristband, a heart rate belt, a helmet, a necklace, a watch, a ring, a bracelet, clothing or a belt, but the invention is not limited thereto. The wearable apparatus 100 includes a sensing module 110, an audio-video module 120 and a control module 130, but the invention is not limited thereto.

In an embodiment of the invention, the sending module 110 may include, for example, an accelerometer, a gravity sensor (G Sensor), a gyroscope, a digital compass, an electronic compass or other similar sensors, or a combination of aforesaid sensors, which can be used to sense an acceleration, an orientation, a tilt angle, a rotation angle or a facing direction of the wearable apparatus 100. The sensing module 110 further includes, for example, a hygrometer, a thermometer, a barometer, an air quality detector, a blood pressure monitor, a blood glucose sensor, a blood oxygen sensor, a hydration sensor, an electrocardiograph (ECG, EKG) apparatus, a pedometer, an EMG (electromyography) sensor or other physiological data sensors, or a combination of the aforesaid devices, but the invention is not limited thereto.

In an embodiment of the invention, the audio-video module 120 may include, for example, a microphone, a camera or a combination of aforesaid devices, but the invention is not limited thereto.

In an embodiment of the invention, the control module 130 may be in form of hardware, firmware, or software or machine-executable program codes stored in a memory to be loaded and executed by a micro processor or a digital signal processor. In form of hardware, the control module 130 may be realized by one single integrated circuit chip, or may be completed by using a plurality of circuit chips, but the invention is not limited thereto. The circuit chips or the integrated circuit chip may be implemented by adopting Application-Specific Integrated Circuit (ASIC) or Field Programmable Gate Array (FPGA). The memory may be, for example, a random access memory, a read-only memory or a flash memory.

After the wearable apparatus 100 is started, the sensing module 110 can sense a physiological information PIS of a user and an environment information EIS of an environment where the user is located, and the audio-video module 120 can continuously capture a video and an audio around the user as an audio-video signal AVS.

In addition, the control module 130 is coupled to the sensing module 110 for receiving the physiological information PIS and the environment information EIS, and is coupled to the audio-video module 120 for receiving the audio-video signal AVS. In particular, the control module 130 can compare the physiological information PIS and the environment information EIS with at least one reference information RFI, wherein the at least one reference information RFI corresponds to at least one event (which may be an accident event or a specific disease event related to the user), respectively. When neither of the physiological information PIS and the environment information EIS matches the reference information RFI, the control module 130 can determine that said event does not occur. Accordingly, the control module 130 can control the audio-video module 120 to perform a cyclic audio-video recording so as to store the received audio-video signal AVS as an audio-video information in a manner of cyclic storage. Herein, the cyclic audio-video recording means that the audio-video information previously stored by the control module 130 will be overwritten by the subsequently obtained audio-video signal AVS after a specific period of time, and that specific period of time is correlated with a storage capacity of the wearable apparatus 100.

By contrast, when at least one of the physiological information PIS and the environment information EIS matches at least one of the reference information RFI, the control module 130 can determine that a corresponding event occurs. Accordingly, the control module 130 preserves and stores the audio-video signal AVS within a period of time before and after a time point (i.e., a trigger time point) at which the event occurs to serve as a preserved audio-video signal. In other words, the control module 130 could preserve and store the audio-video signal AVS within a time interval which is from a first time point before the trigger time point to a second time point after the trigger time point. In particular, the first time point and the second time point could be determined based on the needs of actual use or design requirements. In this way, the preserved audio-video signal would not be overwritten by the audio-video signal AVS subsequently received from the audio-video module 120, so as to facilitate appropriate treatments or determining and clarifying attribution of responsibility in subsequent processes.

In an embodiment of the invention, when the event occurs, the control module 130 can further preserve and store the physiological information PIS and the environment information EIS within the period of time before and after the time point at which the event occurs, so as to facilitate appropriate treatments or determining and clarifying attribution of responsibility in subsequent processes.

In an embodiment of the invention, the environment information EIS may include at least one of a value of gravity acceleration taken by the user, an environmental temperature value, an environmental humidity value, an environmental air pressure value, an ultraviolet value and a particulate matter (PM2.5) value in the environment where the user is located, but the invention is not limited thereto.

For instance, if the environment information EIS is the value of gravity acceleration taken by the user and the value of gravity acceleration taken by the user (sensed by the sensing module 110) matches a reference gravity acceleration value in the reference information RFI, it indicates that the user may encounter the accident event involving falling-down, collision, falling-off the building or the like. Therefore, the control module 130 can determine that the accident event corresponding to the reference gravity acceleration value in the reference information RFI occurs, and preserves and stores the audio-video signal AVS within the period of time before and after the time point at which the accident event occurs. Alternatively, the audio-video signal AVS, the physiological information PIS and the environment information EIS within the period of time before and after the time point at which the event occurs are all preserved and stored to facilitate appropriate treatments or determining and clarifying attribution of responsibility in subsequent processes.

In yet another embodiment, if the environment information EIS is the temperature value in the environment where the user is located and the temperature value in the environment where the user is located (sensed by the sensing module 110) matches a reference temperature value (e.g., above 70° C., but not limited thereto) in the reference info illation RFI, it indicates that there may be the accident event involving fire disaster in the environment where the user is located. Therefore, the control module 130 can determine that the accident event corresponding to the reference temperature value in the reference information RFI occurs, and preserves and stores the audio-video signal AVS within the period of time before and after the time point at which the accident event occurs. Alternatively, the audio-video signal AVS, the physiological information PIS and the environment information EIS within the period of time before and after the time point at which the event occurs are all preserved and stored to facilitate appropriate treatments or determining and clarifying attribution of responsibility in subsequent processes.

In an embodiment of the invention, the physiological information PIS may include at least one of a body temperature value, a blood pressure value, a blood glucose value, a blood oxygen value, a brain wave value, an electrocardiogram, a heart rate value, an electromyography value, a respiratory rate value, a breath chemical composition value and a sweat secretion value of the user, but the invention is not limited thereto.

For instance, if the physiological information PIS is the blood pressure of the user and the blood pressure of the user (sensed by the sensing module 110) matches a reference blood pressure value in the reference information RFI, it indicates that the user may encounter the specific disease event involving hypertension or hypotension. Therefore, the control module 130 can determine that the specific disease event corresponding to the reference blood pressure value in the reference information RFI occurs, and preserves and stores the audio-video signal AVS within a period of time before and after the time point at which the specific disease event occurs. Alternatively, the audio-video signal AVS, the physiological information PIS and the environment information EIS within the period of time before and after the time point at which the event occurs are all preserved and stored to facilitate appropriate treatments or determining and clarifying attribution of responsibility in subsequent processes.

FIG. 2 illustrates a circuit block diagram of a wearable apparatus 200 according to another embodiment of the invention. As similar to the wearable apparatus 100 of FIG. 1, the wearable apparatus 200 of FIG. 2 may also be a wearable electronic apparatus realized in a user-wearable manner including a tag, a wristband, a heart rate belt, a helmet, a necklace, a watch, a ring, a bracelet, a clothing or a belt, but the invention is not limited thereto. The wearable apparatus 200 includes a sensing module 110, an audio-video module 120, a control module 130, a storage module 140, a communication module 150 and a warning module 160, but the invention is not limited thereto. Implementations and operations of the sensing module 110, the audio-video module 120 and the control module 130 of FIG. 2 are similar to those of the sensing module 110, the audio-video module 120 and the control module 130 of FIG. 1 respectively, and thus descriptions regarding the same may refer to related descriptions of FIG. 1, which are not repeated hereinafter. In the following, only the storage module 140, the communication module 150 and the warning module 160 of FIG. 2 are described.

The storage module 140 is coupled to the control module 130 and the audio-video module 120. When the control module 130 determines that an event (e.g., the accident event or the specific disease as described above) occurs, the control module 130 can control the audio-video module 120 to store the audio-video signal AVS within the period of time before and after the time point at which the event occurs into the storage module 140. In this way, the audio-video signal AVS within the period of time before and after the time point at which the event occurs will not be deleted nor overwritten. Moreover, the control module 130 may also store the physiological information PIS and the environment information EIS received within the period of time before and after the time point at which the event occurs into the storage module 140. In an embodiment of the invention, the storage module 140 may be, for example, a hard disk drive (HDD), a volatile memory, a non-volatile memory or a combination of aforesaid devices, but the invention is not limited thereto.

The communication module 150 is coupled to the storage module 140. The communication module 150 can transmit the audio-video signal AVS, the physiological information PIS and the environment information EIS stored in the storage module 140 to an external electronic apparatus (not illustrated) so as to ask for help, but the invention is not limited thereto. In an embodiment of the invention, the communication module 150 may include, for example, a wireless communication module, a wired communication module or a combination of both the wireless communication module and the wired communication module. The wired communication module may be, for example, a Wi-Fi (Wireless Fidelity) module, a Bluetooth module, a BLE (Bluetooth Low Energy) module, an Infrared module, an NFC (Near Field Communication) module, a LTE (Long Term Evolution) module, a WiMax module, a D2D (Device-to-Device) module or a combination of the aforesaid modules. The wired communication module may be, for example, a USB (Universal Serial Bus) interface, a RS232 interface, an UART (Universal Asynchronous Receiver/Transmitter) interface, an I2C (Inter-Integrated Circuit) interface, a SPI (Serial Peripheral Interface) interface, a display port, a Thunderbolt interface, a LAN (Local Area Network) interface or a combination of aforesaid interfaces.

The warning module 160 is coupled to the control module 130. When the event occurs, the control module 130 controls the warning module 160 to send out a warning signal WS. The warning signal WS may be, for example, a signal in form of sound or light so the surrounding people can learn that the user of the wearable apparatus 200 is experiencing an emergency according to the warning signal WS. In an embodiment of the invention, the warning module 160 may be, for example, a buzzer, a speaker, an alarm lamp or a combination of the aforesaid devices.

In an embodiment of the invention, as shown in FIG. 2, the control module 130 may include a data storage module 131. The data storage module 131 is configured to store the at least one reference information RFI, but the invention is not limited thereto. Further, because the reference information RFI may be different based on different persons, the control module 130 may further update the at least one reference information RFI based on a requirement of the user such that wearable apparatus 200 can be more flexible in use. In an embodiment of the invention, the storage module 131 may be, for example, a volatile memory, a non-volatile memory or a combination of aforesaid devices, but the invention is not limited thereto.

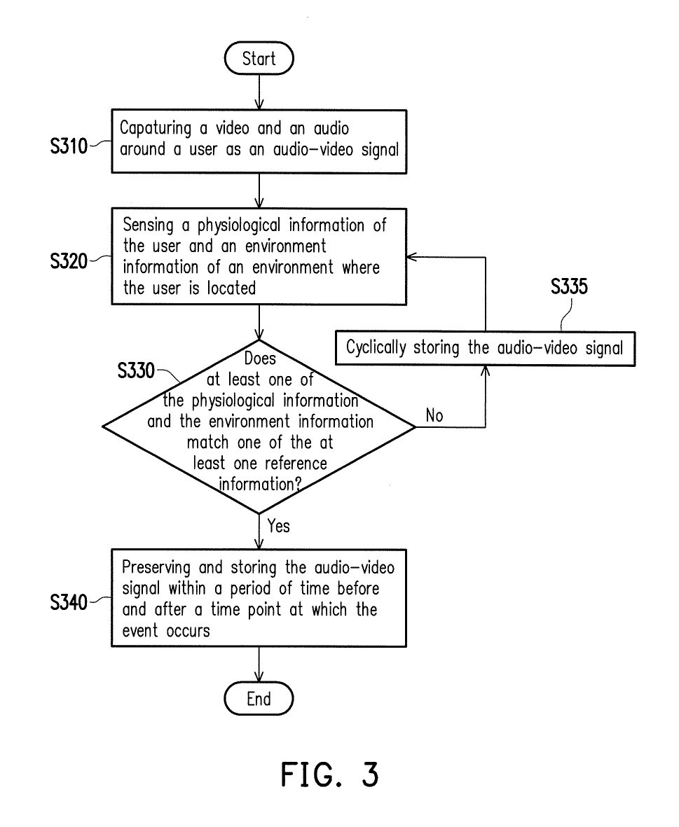

FIG. 3 is a flowchart illustrating steps in an audio-video recording method according to an embodiment of the invention. Referring to FIG. 1 and FIG. 3 together, after wearable apparatus 100 is worn and started by the user, in step S310, a video and an audio around the user can be captured as an audio-video signal AVS by the audio-video module 120 of the wearable apparatus 100. Next, in step S320, a physiological information PIS of the user and an environment information EIS of an environment where the user is located may be sensed by the sensing module 110 of the wearable apparatus 100.

Subsequently, in step S330, the physiological information PIS and the environment information EIS are compared with at least one reference information RFI and whether at least one of the physiological information PIS and the environment information EIS matches one of the at least one reference information RFI is determined by the control module 130 of the wearable apparatus 100. If a comparison result is yes in step S330, it can be determined that an event has occurred, and an audio-video signal AVS within a period of time before and after a time point at which the event occurs can be preserved and stored by the control module 130 as shown in step S340, so as to facilitate appropriate treatments or determining and clarifying attribution of responsibility in subsequent processes. If the comparison result is no in step S330, it is determined that the event does not occur, and the audio-video module 120 is controlled by the control module 130 to perform a cyclic audio-video recording so as to cyclically store the received audio-video signal AVS, as shown in step S335. Next, the method returns to step S320 in which the physiological information PIS of the user and the environment information EIS of the environment where the user is located are re-sensed, and then continues to execute step S330.

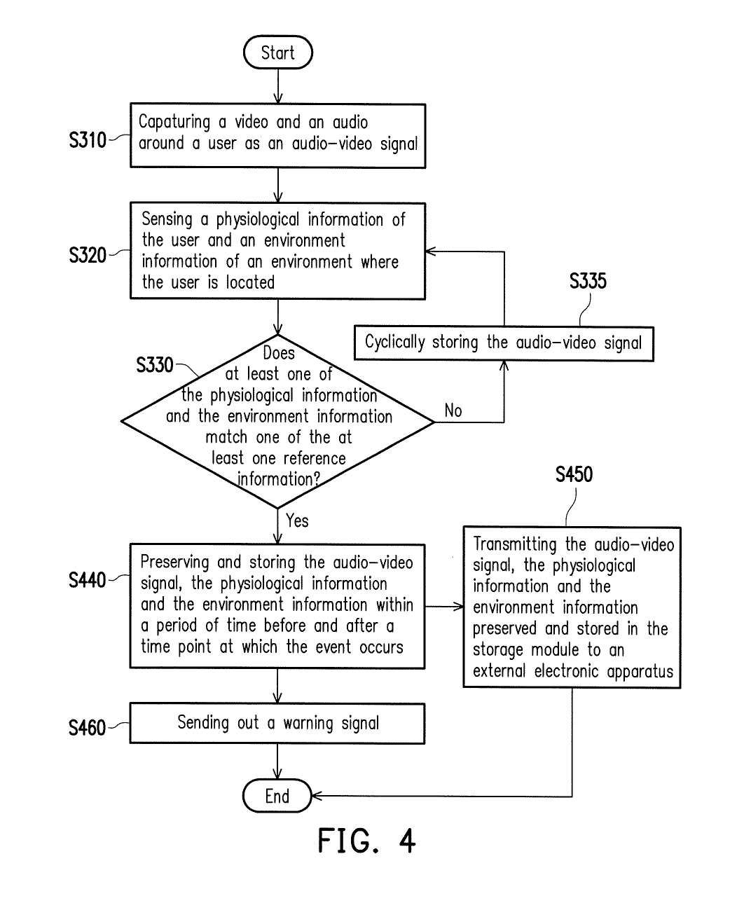

FIG. 4 is a flowchart illustrating steps in an audio-video recording method according to another embodiment of the invention. Referring to FIG. 2 to FIG. 4 together, steps S310, S320, S330 and S335 in FIG. 4 are similar to steps S310, S320, S330 and S335 in FIG. 3 respectively and can refer to related description of FIG. 3, which are not repeated hereinafter.

In addition, if a comparison result is yes in step S330 in FIG. 4, it can be determined that the event has occurred, and the audio-video signal AVS, the physiological information PIS and the environment information EIS within the period of time before and after the time point at which the event occurs can be preserved and stored to the storage module 140 by the control module 130, as shown in step S440, so as to facilitate appropriate treatments or determining and clarifying attribution of responsibility in subsequent processes. Next, in step S450, the audio-video signal AVS, the physiological information PIS and the environment information EIS preserved and stored in the storage module 140 can be transmitted to an external electronic apparatus by the communication module 150, so as to ask for help; and in step S460, a warning signal WS can be sent out by the warning module 160, such that the surrounding people can learn that the user of the wearable apparatus 200 is experiencing an emergency according to the warning signal WS.

In summary, once a specific event occurs on the user of the wearable apparatus, the wearable apparatus and the audio-video recording method thereof of the invention are capable of preserving and storing the information within the period of time before and after the time point at which the specific event occurs, so as to facilitate appropriate treatments or determining and clarifying attribution of responsibility in subsequent processes. In particular, the stored information can include the audio-video signal around the user, the physiological information of the user and the environment information of the environment where the user is located.

It will be apparent to those skilled in the art that various modifications and variations can be made to the structure of the present invention without departing from the scope or spirit of the invention. In view of the foregoing, it is intended that the present invention cover modifications and variations of this invention provided they fall within the scope of the following claims and their equivalents.

What is claimed is:

1. A wearable apparatus, comprising:
a sensing module, configured to sense a physiological information of a user and an environment information of an environment where the user is located;
an audio-video module, configured to capture a video and an audio around the user as an audio-video signal;
a control module, coupled to the sensing module for receiving the physiological information and the environment information, and coupled to the audio-video module for receiving the audio-video signal,
wherein the control module compares the physiological information and the environment information with at least one reference information,
wherein when at least one of the physiological information and the environment information matches one of the at least one reference information, the control module determines that an event occurs and the control module preserves and stores the audio-video signal within a period of time before and after a time point at which the event occurs.

2. The wearable apparatus according to claim 1, wherein when the event occurs, the control module further preserves and stores the physiological information and the environment information within the period of time before and after the time point at which the event occurs,
wherein the environment information comprises at least one of a gravity acceleration value, a temperature value, a humidity value, an air pressure value, an ultraviolet value and a particulate matter value, and the physiological information comprises at least one of a body temperature value, a blood pressure value, a blood glucose value, a blood oxygen value, a brain wave value, an electrocardiogram value, a heart rate value, an electromyography value, a respiratory rate value, a breath chemical composition value and a sweat secretion value.

3. The wearable apparatus according to claim 1, wherein the control module determines that the event does not occur when neither of the physiological information and the environment information matches the at least one reference information, and the control module controls the audio-video module to perform a cyclic audio-video recording so as to cyclically store the received audio-video signal.

4. The wearable apparatus according to claim 1, further comprising:
a storage module, coupled to the control module and the audio-video module, wherein when the event occurs, the control module controls the audio-video module to store the audio-video signal within the period of time before and after the time point at which the event occurs into the storage module, and the control module stores the physiological information and the environment information received within the period of time before and after the time point at which the event occurs into the storage module.

5. The wearable apparatus according to claim 4, further comprising:
a communication module, coupled to the storage module, and configured to transmit the audio-video signal, the physiological information and the environment information stored in the storage module to an external electronic apparatus.

6. The wearable apparatus according to claim 1, further comprising:
a warning module, coupled to the control module, wherein the control module controls the warning module to send out a warning signal when the event occurs.

7. The wearable apparatus according to claim 1, wherein the control module comprises:
a data storage module, configured to store the at least one reference information, wherein the control module updates the at least one reference information based on a requirement of the user.

8. An audio-video recording method of a wearable apparatus, comprising:
capturing a video and an audio around a user as an audio-video signal by an audio-video module of the wearable apparatus;
sensing a physiological information of the user and an environment information of an environment where the user is located by a sensing module of the wearable apparatus;
comparing the physiological information and the environment information with at least one reference information by a control module of the wearable apparatus, and determining whether at least one of the physiological information and the environment information matches one of the at least one reference information to obtain a comparison result; and
determining that an event occurs if the comparison result is yes, and preserving and storing the audio-video signal within a period of time before and after a time point at which the event occurs by the control module.

9. The audio-video recording method according to claim 8, further comprising:
preserving and storing the physiological information and the environment information within the period of time before and after the time point at which the event occurs by the control module when the event occurs.

10. The audio-video recording method according to claim 9, further comprising:
transmitting the audio-video signal, the physiological information and the environment information within the period of time before and after the time point at which the event occurs to an external electronic apparatus by a communication module of the wearable apparatus when the event occurs.

11. The audio-video recording method according to claim 8, further comprising:
sending out a warning signal by a warning module of the wearable apparatus when the event occurs.

12. The audio-video recording method according to claim 8, further comprising:
determining that the event does not occur when neither of the physiological information and the environment information matches the at least one reference information, and controlling the audio-video module by the control module to perform a cyclic audio-video recording so as to cyclically store the received audio-video signal.
